(19) United States
(12) Patent Application Publication
(10) Pub. No.: US 2021/0214428 A1
(43) Pub. Date: Jul. 15, 2021

(54) IMMUNE CHECKPOINT THERAPY

(71) Applicant: Ralf KLEEF, Vienna (AT)

(72) Inventor: Ralf KLEEF, Vienna (AT)

(21) Appl. No.: 17/204,564

(22) Filed: Mar. 17, 2021

Related U.S. Application Data
(62) Division of application No. 16/481,323, filed on Jul. 26, 2019.

(30) Foreign Application Priority Data
Feb. 28, 2017 (EP) 17158412.1

(51) Int. Cl. C07K 16/18; A61P 35/04; A61P 35/00; A61K 31/549; A61K 31/675; A61K 38/20; A61K 39/395; C07K 14/55; C07K 16/28

(52) U.S. Cl. CPC C07K 16/18; A61P 35/04; A61P 35/00; A61K 31/549; A61K 2039/507; A61K 38/2013; A61K 39/3955; C07K 14/55; C07K 16/2827; A61K 31/675

(57) ABSTRACT

Disclosed is (a) an antibody or an antigen-binding portion thereof that specifically binds to and inhibits Programmed Death-1 (PD-1) and/or an antibody or an antigen-binding protein portion thereof that specifically binds to and inhibits Programmed Death-L1 (PD-L1); (b) an antibody or an antigen-binding portion thereof that specifically binds to and inhibits Cytotoxic T-Lymphocyte Antigen-4 (CTLA-4); and (c) Interleukin-2 (IL-2) for use in the treatment of a cancer patient, wherein the body core temperature of said patient is kept at a temperature of 39.0° C. to 40.5° C., preferably of 39.5° C. to 40.5° C. for at least 5 h per day for at least 4, preferably at least 5, consecutive days.

This is a divisional of application Ser. No. 16/481,323, which is the national stage of international application PCT/EP2018/054925, filed Feb. 28, 2018, the entire disclosure of which is hereby incorporated by reference herein.

The present invention relates to the treatment of cancer patients with immune checkpoint inhibitors.

### BACKGROUND OF THE INVENTION

Cancer is associated with global immune suppression of the host. Malignancy-induced immune suppressive effect can be circumvented by blocking different immune checkpoints and tip the immune balance in favour of immune stimulation and unleash cytotoxic effects on cancer cells. Human antibodies directed against immune checkpoint proteins: cytotoxic T lymphocytes antigen-4 (CTLA-4) and programmed death-1 (PD-1), programmed death-ligand (PD-L1), have shown therapeutic efficacy in advanced melanoma and non-small-cell lung cancer and other malignancies. Immune check point blockade antibodies lead to diminished tolerance to self and enhanced immune ability to recognize and eliminate cancer cells. As a class these agents have immune-related adverse events i.a. due to decreased ability of effector immune cells to discriminate between self and non-self (Wolchok et al., N. Eng. J. Med. 369 (2013), 122-133; Callahan et al., Front. Oncol. 4 (2015), article 385; Das et al., J. Immunol. 194 (2015, 950-959; Khan et al., J. Oncology (2015); article ID 847383; Naidoo et al., Ann. Oncol. 26 (2015), 2375-2391).

Therefore, despite the significant improvement of this type of immune therapy, there are still significant problems associated with this therapy, specifically with respect to efficacy/safety issues and (auto-)immune-related adverse effects associated with such checkpoint inhibitor (CI) therapies.

It is the primary object of the present invention to provide new and improved therapies for cancer patients based on CIs, especially therapies with higher efficacy and/or lower adverse effects. A secondary objective of the present invention is to offer effective immunotherapies which are much less expensive than the currently established therapy regimens (The checkpoint inhibitor drugs are expected to become a $30bn-plus market by 2022).

### SUMMARY OF THE INVENTION

Therefore, the present invention relates to (a) an antibody or an antigen-binding portion thereof that specifically binds to and inhibits Programmed Death-1 (PD-1) and/or an antibody or an antigen-binding protein portion thereof that specifically binds to and inhibits Programmed Death-L1 (PD-L1); (b) an antibody or an antigen-binding portion thereof that specifically binds to and inhibits Cytotoxic T-Lymphocyte Antigen-4 (CTLA-4); and (c) Interleukin-2 (IL-2) for use in the treatment of a cancer patient, wherein the body core temperature of said patient is kept at a temperature of 39.0° C. to 40.5° C., preferably of 39.5° C. to 40.5° C. for at least 5 h per day for at least 4, preferably at least 5, consecutive days.

The present invention provides a CI therapy wherein a PD1/PD-L1 inhibiting agent (preferably an PD-1/PD-L1 antibody or a PD-1/PD-L1 binding portion thereof) and a CTLA-4 inhibiting agent (preferably a CTLA-4 antibody or a CTLA-4 binding portion thereof) is administered to a patient, preferably in a lower dose than the doses currently applied (see e.g. Wolchok et al., 2015), together with IL-2, wherein the IL-2 administration is not accompanied with antipyretics (as required by the administration leaflets) but wherein the patient is allowed to develop controlled significant fever (i.e. at least 5 h over 39.0° C., preferably over 39.5° C. for at least 4, preferably at least 5, consecutive days).

### BRIEF DESCRIPTION OF THE DRAWINGS

FIG. 1A shows the body core temperature of the cancer patient of case 1;

FIGS. 1B and 1C show endoscopies before immunotherapy (month 0) demonstrating exulcerated advanced T4 esophageal cancer;

FIGS. 1D and 1E show: endoscopy following immunotherapy (month 2) according to the present invention demonstrating complete remission.

FIG. 2 shows the body core temperature of the cancer patient of case 2.

FIG. 3 shows the body core temperature of the cancer patient of case 3.

FIG. 4A-D shows the body core temperature of the cancer patient of case 4 at four exemplary consecutive days; FIGS. 4E-J show pictures (pre/after treatment) of skull (E, F), abdomen/pelvis (G, H) and chest (I, J) following therapy according to the present invention demonstrating complete remission; FIG. 4K shows inhibition of chemotherapeutic compounds for this patient.

### DETAILED DESCRIPTION OF THE INVENTION

The presented concept and invention targeting the induction of fever in cancer patients is a modern adaptation of a concept for immunotherapy of cancer that started 130 years ago with William Coley (Kleef et al. Neuroimmunomodulation. 2001; 9(2):55-64. Review.) and that has been adapted with surprising effectiveness to modern immunotherapy by the present invention.

Whereas CI therapies have applied IL-2 in the past (Kleef et al., ASCO (2016), Abstract 166013 (Kleef et al., J. Clin. Oncol. 34 (2016): e33111); Wolchok et al., 2015; Khan et al., 2015), fever was induced mainly by whole body or local hyperthermia devices with 13.56 MHz modulated radiofrequency (e.g. Oncotherm, Synchrotherm, etc.). Such methods are also applied in connection with chemotherapy and radiation therapy in cancer patients. Although such hyperthermia treatments have been successfully applied (and are even preferably applied in the methods according to the present invention in addition to the basic fever treatment according to the present invention), the methods and treatments according to the present invention have shown significantly better success rates by imposing a longer (at least 4, preferably at least 5, consecutive days instead of (only) one radiofrequency treatment or more than one radiofrequency treatment timely separated) and more intense fever induction (at least 5 h per day, preferably at least 6 h per day, more preferred at least 7 h per day, especially at least 8 h per day) for at least 4, preferably at least 5, consecutive days) on the patient. It is also possible—if the patient's condition allows—to keep linger fever durations per day (at least 10 h or at least 12 h), however, such longer circles have not been proven yet to gain additional benefit and have also practical drawback, since the patients also need to recover from the fever circles. Accordingly, the fever treatments should also be optimised within the borderlines of the present invention to take the individual condition of the patient before and during the treatment into consideration.

The daily fever induction is necessary to be performed at least for 4, preferably at least 5, consecutive days. It is also possible to perform the induction of at least 39.0° C., preferably of 39.5° C. to 40.5° C., per diem for at least 6 consecutive days, preferably for at least 7 consecutive days, especially for at least 8 consecutive days (e.g. for 9, 10, 11, or 12 consecutive days).

It is important according to the present invention to keep the patients at a temperature level of at least 39.0° C., preferably of 39.5° C. to 40.5° C., i.e. at high fever at least once a day for an effective time range, to achieve the results according to the present invention. Lower temperatures or less duration (e.g. two or three days only) will not achieve the efficacy of the present invention. Higher fever (up to 41.0° C.) is in principle not excluded, however, care should be taken that the patient is kept in a condition wherein the fever is not life-threatening. This is why the aimed range of preferably 39.0° C. to 40.5° C. has shown to be the balanced range with respect to risk/efficacy for the present invention.

The fever according to the present invention is preferably imposed on the patient by administering IL-2 in a way so as to achieve the aimed temperature range. The patients may therefore be “IL-2 titrated” with respect to the fever, i.e. the fever may be induced and controlled simply by the IL-2 dosage, however, without co-administration of fever-reducing agents (antipyretics) which are usually required as IL-2 co-medication (see product leaflet for the commercial IL-2 product PROLEUKIN® (aldesleukin) recommending standard antipyretic therapy (especially NSAID therapy immediately before IL-2 therapy as concomitant medications; see also e.g. Dutcher et al., J. Immunother. Cancer 2 (2014), 26). According to a preferred embodiment of the present invention, body temperature of the patient is controlled by administration of IL-2 (“IL-2 fever titration”; “IL-2 induced fever”).

The present invention preferably applies the IL-2 treatment with lower doses than usually recommended, especially in connection with CI therapy: Whereas usual IL-2 administration applies in total well over 500 million units IL-2/week, the overall amount of IL-2 administered in the course of the present invention is preferably in the range of 40 to 70 million units IL2/week, especially in the range of 45 to 60 million units IL-2. Other preferred ranges for such low dose IL-2 treatment are 20 to 250 million units IL-2/week, preferably 40 to 200 million units IL-2/week, more preferred 50 to 100 million units IL2/week, especially 60 to 80 million units IL-2/week (or any range defined by these borders).

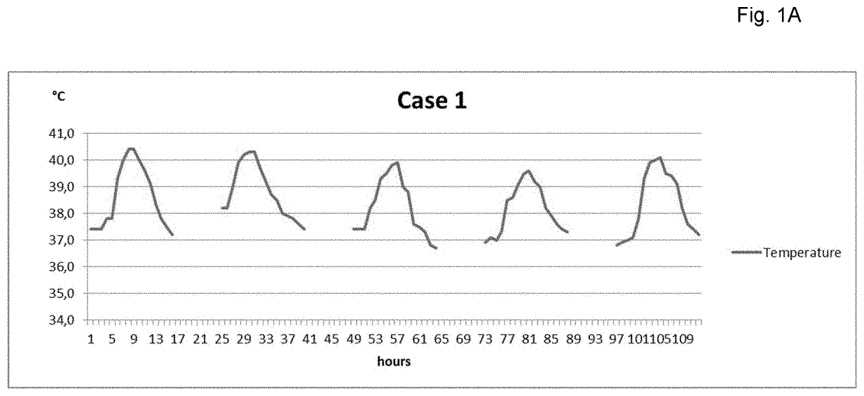

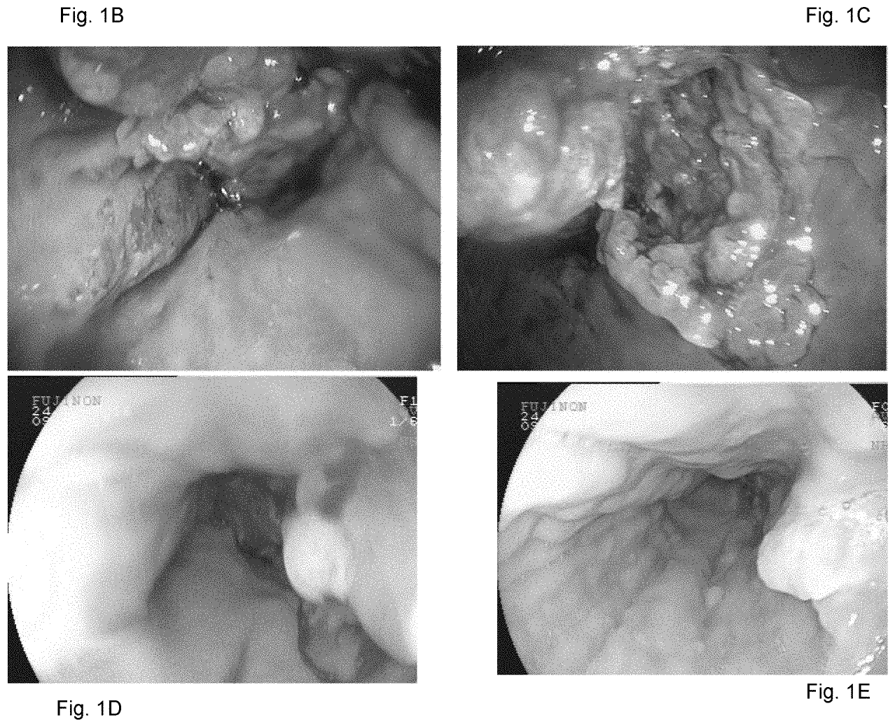

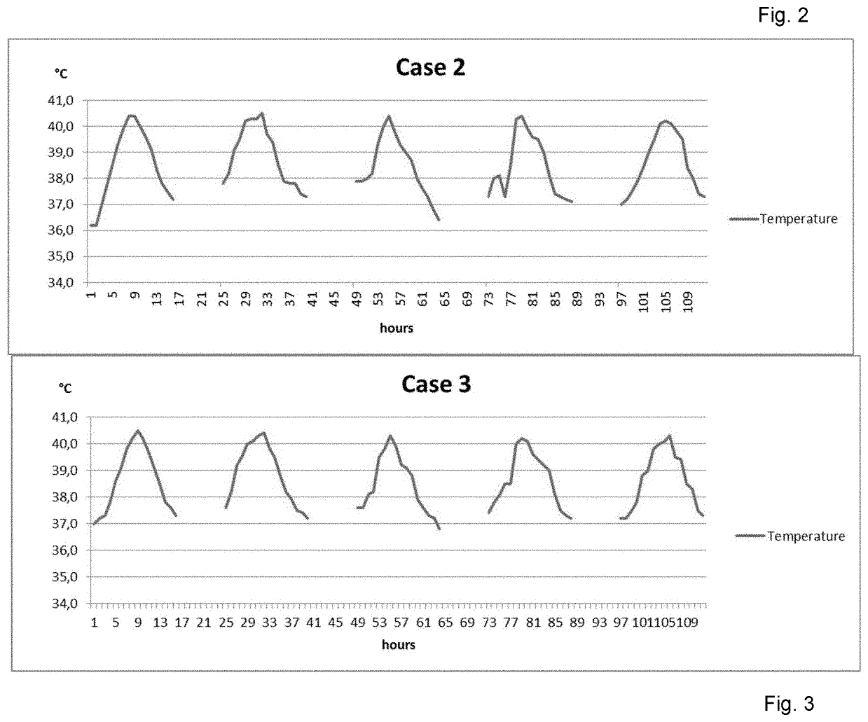

It is specifically preferred to administer IL-2 in an amount sufficient to keep an appropriate, but not life-threatening fever temperature of the patient, i.e. temperatures of 39.0° C. to 40.5° C., preferably of 39.5° C. to 40.5° C. for at least 5 h per day for at least 4, preferably at least 5, consecutive days. It is preferred to administer IL-2 so as to obtain fever developments in the patient according to or similar to the fever curves in FIGS. 1A, 2 and 3, i.e. arriving at least once a day at a (maximum) temperature of between 39.5 to 41.0° C., preferably obtaining at least once a day a temperature of above 40° C. The fever in the patient should be kept for at least 5, preferably at least 6, more preferred at least 7, especially at least 8 h, each of the 4, preferably at least 5, days at a temperature above 39.5° C. by this low dose IL-2 treatment. A “week” is usually a treatment duration form a given day in the week (e.g. Monday) to the same day in the next week. Usually, the treatment regime starts at Monday and extend to Friday (i.e. 4, preferably 5, consecutive working days, often depending on the clinical situation with the last day reserved for post intervention monitoring). However, alternatively (and for practical reasons), the present invention may also be performed in consideration of a weekend, so that the treatment in “4, preferably at least 5, consecutive days” may also be performed in “4, preferably at least 5, consecutive working days” (i.e. excluding treatments of Saturdays and Sundays). This is common in most treatment regimen, especially in the tumour treatment practice. Although the “week” therefore has a duration of 7 days, treatment is usually performed only on the working days in the week, i.e. all days except Saturday and Sunday. Accordingly, the treatment “week” usually comprises 4, preferably at least 5, days wherein medicaments (e.g. IL-2) are administered; however, the amounts of IL-2 disclosed herein for “weekly” administration therefore complies with this duration/administration (i.e. 4, preferably at least 5, days administration in a seven-day time range). The present invention may be applied at least once for 4, preferably at least 5, days. However, the treatment week may also be repeated if necessary. This means that a further 4, preferably 5, consecutive treatment days may be following the initial treatment week. In some instances, an interval may be foreseen between two treatment weeks, e.g. an interval of one, two, three or four weeks or even one, two, three or four months, also depending on the development of the tumour disease in the specific patient. Accordingly, the present IL-2 treatment may be administered for one, two, three or four weeks, preferably for one or two weeks, especially for one week.

Usually IL-2 doses are administered according to a units/m² scheme, i.e. depending on the body surface area (BSA). Each patient has therefore a specific treatment regime. There are several ways to calculate or determine BSA, mostly depending on body weight and height. In cases of doubt, the appropriate BSA formula applied for the present invention shall be Du Bois formula. On average, male patients have a BSA of 1.9 m² and female patients of 1.6 m².

Accordingly, the daily IL-2 dosage may be from 5 to 50 million units, preferably from 10 to 30 million units, especially if daily dosages should be kept constant. Other treatment regimens according to the present invention may also apply different regimen, e.g. with higher starting doses and lower doses in the forthcoming days. For example, 40 to 100 million units IL-2 may be administered on the first day and only half or a quarter of such a dose in the next days. As another example, 40 to 100 million units IL-2 may be administered on the first day, 20 to 50 million units IL-2 may be administered on the second day, and 10 to 30 million units IL-2 may be administered on the third, the fourth and the fifth day. According to a preferred embodiment, IL-2 is administered by continuous administration of 100.000 to 1.000.000 units/kg body weight, preferably 400.000 to 720.000 units/kg body weight, especially as an initial induction of the fever according to the present invention. The following repeated fever cycles can be safeguarded (“titrated”) with lower IL-2 doses, depending on the response of the patient.

Consequently, it is preferred to omit any antipyretic medication in the course of the IL-2 administration according to the present invention or at least reduce such medication to not impede the development of fever in the desired temperature range. Therefore, a preferred embodiment of the present invention involves a treatment which does not include administration of an antipyretic, especially a non-steroidal anti-inflammatory drug (NSAID).

The CI therapy according to the present invention may be performed by any suitable CTLA-4/(PD-1/PD-L1) inhibitor couple. Such inhibitors are widely available in the present field.

For example, WO 2013/173223 A1 discloses antibodies (“Abs”) specific for (and inhibiting) PD-1/PD-L1 and CTLA-4 (see also e.g. U.S. Pat. No. 8,008,449 B2 and U.S. Pat. No. 7,943,743 B2).

Preferred anti-PD-1/PD-L1 HuMAbs exhibit one or more of the following characteristics: (a) binds to human PD-1 with a KD of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumour cell growth in vivo. Anti-PD-1 Abs of the present invention include mAbs that bind specifically to human PD-1 and exhibit at least one, preferably at least five, of the preceding characteristics. U.S. Pat. No. 8,008,449 B2 exemplifies seven anti-PD-1 HuMAbs: 17D8, 2D3, 4H1, 5C4 (also referred to herein as nivolumab or BMS-936558), 4A1 1, 7D3 and 5F4. Isolated DNA molecules encoding the heavy and light chain variable regions of these Abs have been sequenced, from which the amino acid sequences of the variable regions were deduced and disclosed e.g. in WO 2013/173223 A1. Preferred PD-1 inhibitors according to the present invention are mainly nivolumab (Opdivo), and also pembrozilumab (Keytruda), avelumab and atezolizumab (Tecentric).

Preferred anti-CTLA-4 antibodies of the present invention can bind to an epitope on human CTLA-4 so as to inhibit CTLA-4 from interacting with a human B7 counter receptor. Because interaction of human CTLA-4 with human B7 transduces a signal leading to inactivation of T-cells bearing the human CTLA-4 receptor, antagonism of the interaction effectively induces, augments or prolongs the activation of T cells bearing the human CTLA-4 receptor, thereby prolonging or augmenting an immune response. Anti-CTLA-4 antibodies are described e.g. in WO 01/14424 A and WO 00/37504 A; An exemplary clinical anti-CTLA-4 antibody is human monoclonal antibody ipilimumab (WO 2013/173223 A1; Hodi et al., N. Eng. J. Med. 363 (2010), 711 to 723; Wolchok et al., 2015). A further example is tremelimumab. An overview over further suitable anti-CTLA-4 antibodies for use in the therapy according to the present invention is disclosed in U.S. Pat. No. 8,008,449 B2.

Preferably, the anti-CTLA-4 antibody binds to human CTLA-4 with a KD of 5×10⁻⁸ M or less, binds to human CTLA-4 with a KD of 1×10⁻⁸ M or less, binds to human CTLA-4 with a KD of 5×10⁻⁹ M or less, or binds to human CTLA-4 with a KD of between 1×10⁻⁸ M and 1×10⁻¹⁰ M or less.

In contrast to the high doses of antibodies which are usually applied for CI with combined anti-PD-1 and anti-CTLA-4 inhibitors or antibodies (see e.g. Wolchok et al., 2015), it is preferred according to the present invention to use lower doses of such antibodies. The reason for this is twofold: 1) it circumvents the well-known often severe safety issues due to immune-related adverse effects irAE induced by normal dosed checkpoint inhibitor is and 2) the previously never described combination of low-dose checkpoint inhibitors together with low-dose IL-2 allows a synergy effectively mediating antitumor immune response never described before.

Accordingly, a preferred embodiment of the present invention applies a CI therapy with each antibody (PD-1, CTLA-4) being administered at a dosage ranging from 0.05 to 1 mg/kg body weight, preferably from 0.1 to 0.8 mg/kg body weight, especially from 0.3 to 0.5 mg/kg body weight, at least once a week, for at least two weeks, preferably for at least three weeks, especially at least four weeks.

Preferably, the PD-1 antibody is administered in slightly higher doses than the CTLA-4 antibody, especially in the case of nivolumab as PD-1 antibody and ipilimumab as CTLA-4 antibody. Accordingly, the following dosages of PD-1 antibody/CTLA-4 antibody, especially nivolumab/ipilimumab are specifically preferred: 0.1 to 1 mg/kg PD-1 with 0.05 to 0.8 mg/kg; CTLA-4, even more preferred 0.3 to 0.7 mg/kg PD-1 with 0.1 to 0.5 mg/kg CTLA-4, especially 0.4 to 0.6 mg/kg PD-1 with 0.2 to 0.4 mg/kg CTLA-4.

According to another preferred embodiment, cyclophosphamide is administered additionally to the patient in the course of the present invention, preferably in an amount of 100 to 500 mg/m², especially in an amount of 200 to 400 mg/m². Appropriate dosages and amounts of cyclophosphamide may be individually determined based on the individual patient to effectively down modulate T_{reg} cells in this patient at the relevant stage of therapy. The induction of T_{reg} is a well-known mechanism of the body in reaction to IL-2 therapy. It is well known that the induction of T_{reg} induces immune suppressive counter regulatory mechanisms.

In addition, also antimicrobial agents, especially taurolidine, may preferably be administered additionally to said patient.

According to another aspect, the present invention refers to a(ny) checkpoint therapy (CT; CI therapies) for use in a tumour patient, wherein the body core temperature of said patient is kept at a temperature of 39.0° C. to 40.5° C., preferably of 39.5° C. to 40.5° C. for at least 5 h per day for at least 4, preferably at least 5, consecutive days. The therapy principle according to the present invention, although proven to be successful in the treatment of combined inhibition of PD-1 and CTLA-4 (i.e. wherein PD-1 and CTLA-4 inhibitors, especially antibodies, are administered in combination), is also applicable to all treatment regimen wherein immune checkpoints are addressed (inhibited), i.e. which target regulatory pathways in T cells to enhance antitumor immune responses. Practical examples for such CI therapies wherein the principle of the present invention can be applied are disclosed e.g. in Topailan et al., Cancer Cell 27 (2015), 450-461). Antibodies used in the course of such CI therapies and wherein the present invention can be included in the therapy schemes with such antibodies or combination of antibodies are nivolumab (Opdivo, BMS), and ipilimumab (Yervoy, BMS), as disclosed above, but also other antibodies being specific for (inhibiting) immune checkpoint proteins, such pembrolizumab (Keytruda, MK-3475, Merck), pidilizumab (CT-011, Cure Tech), BMS-936559 (anti-PD-1 receptor), atezolizumab (MPDL3280A, Roche), avelumab (Merck KGaA, Darmstadt, Germany & Pfizer) (anti-PD-L1 receptor), and/or tremelimumab (anti CTLA-4)(see also: Ott et al., J. Immunother. Cancer 5 (2017), 16).

Fever induction by the present invention is based on the administration of IL-2, however, this administration is performed by using much lower doses than those doses that have usually been applied in the IL-2 treatment of cancer patients. Whereas cancer patients have been treated with “high dose IL-2 treatments” (“HD IL-2 treatment”) as in Kleef et al. (ASCO (2016), Abstract 166013), longer lasting fever that could have been caused by IL-2 treatment was prevented by co-administration of fever-reducing agents (antipyretics). These antipyretics were mandatorily applied in the treatment of commercial IL-2 products, such as Proleukin® (Aldesleukin). In contrast to these previously applied HD IL-2 dosages, the method according to the present invention uses a “low dose” IL-2 treatment, therefore making the fever controllable and permanent without the addition of antipyretics and without endangering the cancer patients by HD IL-2 treatments (which cannot be performed without antipyretics). The low dose IL-2 treatment of the present invention is characterised by significantly less amount of IL-2 than in usual IL-2 treatments so that it can be administered without antipyretics (without seriously endangering the patient by IL-2) but high enough to obtain the appropriate fever temperature as required for the present invention. For example, an optimal IL-2 low dose treatment is in the range of 40 to 70 million units IL2/week, which is about one order of magnitude in units under the IL-2 treatment prescribed for commercial IL-2 products.

The fact that in the prior art significantly higher doses of IL-2 have been applied (which necessitate the co-administration of antipyretics) is also clear in Kleef et al. (ASCO (2016), Abstract 166013), wherein a “HD IL-2 (54 Mio/m² as decrescendo regimen) therapy” was performed for five days. This complies with the prescribing information for IL-2 being defined by 600,000 units/kg every eight hours over five days. This means that a 50 year old female patient under standard IL-2 dosage having approximately 50 kg and around 1.7 m² body surface is subjected to a daily dose of 54 Mio/m² (if performed for five days, more than 250 Mio I.U. IL-2/m² have been administered to this patient, resulting in a total of more than 450 Mio I.U. IL-2), which is indeed a “high dose” (“HD”) IL-2 treatment as prescribed in the prescribing information for the IL-2 products on the market. This makes also clear that a HD IL-2 treatment was never performed without antipyretic protection and therefore mandatorily prescribed.

The present low dose IL-2 administration is performed to keep the body core temperature of the patient in a “fever state”, i.e. at a temperature of 39.0° C. to 40.5° C., whereas the HD IL-2 treatment was performed with antipyretic protection (i.e. with no long duration fever or at least not with a body temperature of 39.0° C. or higher; see the Dutcher et al. review (2014)).

It is also important to consider that the present low dose IL-2 induced fever is significantly different to the technique of “whole body hyperthermia”. In fact, such “whole body hyperthermia” is usually performed in parallel to moderately dosed chemotherapy with cyclophosphamide. Such a treatment may also be performed within the treatment regimen according to the present invention (in addition to the low dose IL-2 treatment), however, it is preferably performed before the low dose IL-2 treatment according to the present invention. The rationale of such “whole body hyperthermia” treatment preceding the IL-2 treatment of the present invention is the prophylactic down-modulation of immunosuppressive T_{reg} cells. This external fever induction is completely different from the internal fever induction according to the present invention and is performed by using special medical devices with water-filtered infrared-A radiation. This external fever induction significantly differs from the fever induction according to the present invention (Repasky et al., Cancer Immunol. Res. 1 (2013), 210-216; WO 00/28813 A1). Such external fever interaction is therefore suitable to be performed at a different stage of the procedure (with the chemotherapy) and for a different purpose (to downmodulate T_{reg} cells).

It is also remarkable to note that there is no indication whatsoever in the prior art to perform a low dose IL-2 treatment with the proviso that the body core temperature of the patient is kept at a temperature of 39.0° C. to 40.5° C. instead of the HD IL-2 treatment as applied previously and performed under antipyretic protection. The way in which the present invention developed, the IL-2 treatment in tumour patients was contrary to all treatment regimens for IL-2 in tumour patients so far and differs not only in the fact that low amounts of IL-2 were administered, but also that these low dose IL-2 administrations were performed in order to elicit a controlled fever in the patient, i.e. to allow the “side reaction” fever and not suppressed, long lasting fever by antipyretics as prescribed in the prescribing information for IL-2.

Specifically preferred embodiments include the following embodiments:

1. An (a) antibody that specifically binds to and inhibits Programmed Death-1 (PD-1) and/or an antibody that specifically binds to and inhibits Programmed Death-L1 (PD-L1); (b) an antibody that specifically binds to and inhibits Cytotoxic T-Lymphocyte Antigen-4 (CTLA-4); and (c) Interleukin-2 (IL-2) for use in the treatment of a cancer patient, wherein the body core temperature of said patient is kept at a temperature of 39.0° C. to 40.5° C., preferably of 39.5° C. to 40.5° C. for at least 5 h per day for at least 4, preferably at least 5, consecutive days, wherein the overall amount of IL-2 administered is preferably in the range of 40 to 70 million units IL-2/week.

2. An (a) antibody that specifically binds to and inhibits PD-1 and/or an antibody that specifically binds to and inhibits PD-L1; (b) an antibody that specifically binds to and inhibits CTLA-4; and (c) IL-2 for use according to embodiment 1, wherein the body core temperature of said patient is kept at a temperature of 39.0° C. to 40.5° C., preferably of 39.5° C. to 40.5° C. for at least 5 h per day, preferably at least 6 h per day, preferably at least 7 h per day, especially at least 8 h per day, for at least 4, preferably at least 5, consecutive days.

3. An (a) antibody that specifically binds to and inhibits PD-1 and/or an antibody that specifically binds to and inhibits PD-L1; (b) an antibody that specifically binds to and inhibits CTLA-4; and (c) IL-2 for use according to embodiment 1 or 2, wherein the body core temperature of said patient is kept at a temperature of 39.0° C. to 40.5° C., preferably of 39.5° C. to 40.5° C. for at least 5 h per day for at least 6 consecutive days, preferably for at least 7 consecutive days, especially for at least 8 consecutive days.

4. An (a) antibody that specifically binds to and inhibits PD-1 and/or an antibody that specifically binds to and inhibits PD-L1; (b) an antibody that specifically binds to and inhibits CTLA-4; and (c) IL-2 for use according to any one of embodiments 1 to 3, wherein the body temperature of said patient is controlled by administration of IL-2.

5. An (a) antibody that specifically binds to and inhibits PD-1 and/or an antibody that specifically binds to and inhibits PD-L1; (b) an antibody that specifically binds to and inhibits CTLA-4; and (c) IL-2 for use according to any one of embodiments 1 to 4, wherein the treatment does not include administration of an antipyretic, especially a non-steroidal anti-inflammatory drug (NSAID).

6. An (a) antibody that specifically binds to and inhibits PD-1 and/or an antibody that specifically binds to and inhibits PD-L1; (b) an antibody that specifically binds to and inhibits CTLA-4; and (c) IL-2 for use according to any one of embodiments 1 to 5, wherein IL-2 is administered by continuous administration of 100.000 to 1.000.000 units/kg body weight, preferably 400.000 to 720.000 units/kg body weight.

7. An (a) antibody that specifically binds to and inhibits PD-1 and/or an antibody that specifically binds to and inhibits PD-L1; (b) an antibody that specifically binds to and inhibits CTLA-4; and (c) IL-2 for use according to any one of embodiments 1 to 6, wherein, independently of each other, each antibody is administered at a dosage ranging from 0.05 to 1 mg/kg body weight, preferably from 0.1 to 0.8 mg/kg body weight, especially from 0.3 to 0.5 mg/kg body weight, at least once a week, preferably at least twice a week, especially at least three times a week, for at least two weeks, preferably for at least three weeks, especially at least four weeks.

8. An (a) antibody that specifically binds to and inhibits PD-1 and/or an antibody that specifically binds to and inhibits PD-L1; (b) an antibody that specifically binds to and inhibits CTLA-4; and (c) IL-2 for use according to any one of embodiments 1 to 7, wherein cyclophosphamide is administered additionally to said patient, preferably in an amount of 100 to 500 mg/m², especially in an amount of 200 to 400 mg/m².

9. An (a) antibody that specifically binds to and inhibits PD-1 and/or an antibody that specifically binds to and inhibits PD-L1; (b) an antibody that specifically binds to and inhibits CTLA-4; and (c) IL-2 for use according to any one of embodiments 1 to 8, wherein taurolidine is administered additionally to said patient.

The present invention is further described by the following examples, yet without being restricted thereto.

### Examples

Further case reports referring to Immunotherapy in stage IV cancer patients using moderate dosed IL-2 for fever induction in combination with low-dose checkpoint inhibitors and hyperthermia

Case 1: Complete Response of Stage IIIB Esophageal Cancer Combining Low-Dose Checkpoint Inhibitors with Interleukin-2 (IL-2) and Fever Range Hyperthermia.

Advanced stage inoperable esophageal cancer has a poor prognosis and patients rarely enjoy durable complete response to treatment; progression free survival often is limited.

### Materials and Methods:

The patient was a 56-year-old male newly diagnosed with adenocarcinoma of the esophagus with mediastinal lymphadenopathy. Histology revealed adenocarcinoma stage UICC IIIB T4 N2 with disseminated mediastinal, para-esophageal and cervical lymph node metastasis measuring up to 2.2 cm. HER-2/new score was positive. The patient refused suggested neoadjuvant CHT with FLOT. Clinically the patient presented with Karnofsky index of 90% with increasing difficulties swallowing solid food and rapid weight loss of 6 kg in the last 2 months.

Therapy started consisted of administration of the following combination protocol: Low-dose PD-1 immune checkpoint (IC) inhibitor nivolumab (0.5 mg/kg) with CTLA-4 IC inhibitor ipilimumab (0.3 mg/kg) administered weekly, over three weeks. This was accompanied by loco regional hyperthermia with radiofrequency fields (13.56 MHz) using the Syncrotherm device 3 times per week (max output 400 w) over the tumor region in combination with high dose vitamin C (0.5 g/kg) and alpha lipoic acid (600 mg) over three weeks. This was followed by long duration fever range whole body hyperthermia (using the Heckel device) in combination with low dose chemotherapy using cyclophosphamide 300 mg/m² to down modulate T_{reg} cells. Next, moderate dose i.v. interleukin 2 (IL-2) under Taurolidine protection was administered for five consecutive days with careful titration to daily fever hyperthermia of max 39.5°-40.5° C. IL-2 dosage in total over 5 days was 5 Mio/m² daily resulting in a total dosage of 50 million IL-2 over 5 days. Herceptin in a dosage of only 4 mg/kg (patient refused higher dosage) was administered once. The body core temperature of the cancer patient during the treatment of the present invention is depicted in FIG. 1.

### Results:

Unexpectedly, restaging 8 weeks following initiation of therapy with Gastroesophagoscopy (Upper GI Endoscopy) revealed complete response. This was confirmed by histological analysis of multiple biopsies in the former tumour bed confirming complete pathological response. At that time the patient had started gaining weight again and was free of any cancer-related symptoms. Several months later, he has regained 6 kg of weight, feels good, has no dysphagia, and continues to be monitored. FIGS. 1B and 1C show endoscopies before immuno-therapy (month 0) demonstrating exulcerated advanced T4 esophageal cancer; FIGS. 1D and 1E show: endoscopy following immunotherapy (month 2) according to the present invention demonstrating complete remission. Current follow-up time demonstrating lasting complete remission at the time of submission of this paper is 16 months.

### Conclusion:

This advanced stage cancer patient therefore had a surprising and completely unexpected complete response to primary immunotherapy treatment.

Case 2: Complete Clinical Remission of Stage IV Inoperable Prostate Cancer Combining Low-Dose Checkpoint Inhibitors with Interleukin-2 (IL-2) and Fever Range Hyperthermia

Inoperable stage IV advanced prostate cancer following R1 resection has a poor prognosis.

The patient was a 58 old year old male first diagnosed July 2016 when he underwent radical prostatectomy and lymphadenectomy on Aug. 23, 2016 in Germany for locally advanced prostate cancer with infiltration of the sphincter muscle pN1 (3/13), G3, R1 Gleason score 9, ISUP Grading WHO: 5. Initially the patient had severe obstructive urine flow and needed urgent treatment. Radiotherapy and permanent catheter was not consented.

One month post-operative MRI restaging of abdomen and small pelvis from 20 Sep. 2016 demonstrated a lesion left posterior of the bladder neck of 1.3×1.2×2 cm inseparable from the left lateral rectal wall and adjoining portion of pubococcegeus portion of the left levator ani. This soft tissue mass well explained the severe small pelvis pain the patient was suffering from.

The suggested palliative hormonal therapy and radiation was not initiated yet. Radical resection including sphincter resection with permanent colostomy was rejected.

ND: 10 years ago the patient was diagnosed with hepatitis C which was cured 2013 by triple combination therapy of ribarafin, interferon and sovalvir.

The patient presented with Karnofsky Index of 80%, left sided deep pain in the small pelvis VAS9 (!) treated with NSAR, severe incontinence. Weight loss of 10 kg following surgery.

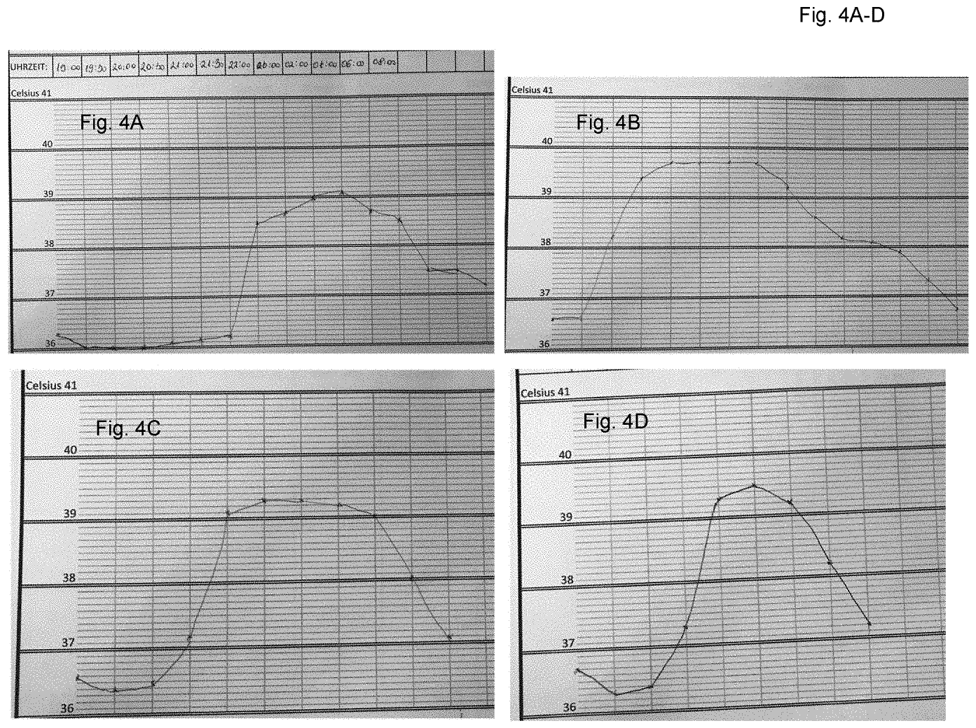

The patient underwent the same treatment concept as described in detail in case #1 with the addition of hormone therapy with Trenantone/Casodex and metronomic low-dose chemotherapy and without Herceptin. The body core temperature of the cancer patient during the treatment of the present invention is depicted in FIG. 2.

### Results:

Unexpectedly restaging with MRI of the abdomen and small pelvis at the end of February 2017 revealed complete remission; the patient is in excellent health and has no tumor-associated symptoms or ailments.

Case 3: Complete Clinical Remission of Stage IV Colon Cancer Combining Low-Dose Checkpoint Inhibitors with Interleukin-2 (IL2) and Fever Range Hyperthermia

Metastatic stage IV colon cancer has a poor prognosis with very limited overall survival and limited therapeutic options. The patient was a 45 year old female first diagnosed with metastatic colon cancer in May 2013 cT3 cN2 cM1b (liver and lung). Histology revealed ulcerated low differentiated adenocarcinoma of enteral type, K-RAS mutation exon 2: Substitution of p.G12V, no microsatellite instability. Further staging proved locally advanced subtotal proximal rectal cancer with multiple local regional lymph node metastases, liver metastasis and disseminated lung metastasis of up to 3.5 cm.

The patient underwent multiple palliative systemic chemotherapies with FOLFOX and Avastin which induced partial remission followed by Avastin maintenance therapy. PET staging in May 2015 showed PD in the rectal area as well as pulmonic PD but complete remission of liver metastasis. June-July 2015 the patient underwent surgical resection of the primary colon cancer with transient colostomy. September 2015 massive progression of lung metastasis was diagnosed.

The patient presented with Karnofsky Index of 90%, mild shortness of breath on exertion.

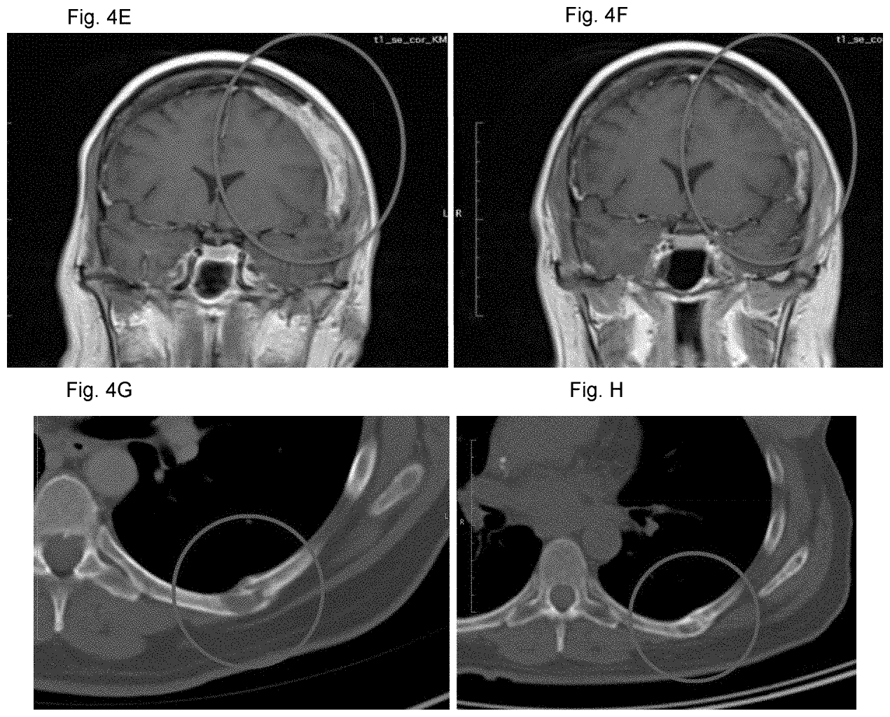

The patient underwent the same treatment concept as described in detail in case #1. The body core temperature of the cancer patient during the treatment of the present invention is depicted in FIG. 3.

Interestingly, initial restaging 5 months later indicated progressive disease of 2 lung metastasis which were clinically presenting with increased coughing. The patient therefore underwent radiation therapy to the pulmonic-mediastinal area.

### Results:

Unexpectedly restaging with CT of the thorax and abdomen at the end of June 2016 revealed complete remission of previously described progressive (DD: pseudo-progression) lung metastasis; the patient is in excellent health and has no tumor-associated symptoms or ailments.

### Conclusion:

The intermittent PD of the patient's lung metastasis 5 months following our treatment concept may be interpreted as so-called pseudo-progression. It is realized by now that immune therapies exert their effects on cancer indirectly by building an immune response first, which is then followed by changes in tumor burden or patient survival. The median time to achieve complete response was 30 months. Immune therapy may induce unusual kinetics of antitumor response, which is not captured by Response Evaluation Criteria in Solid Tumors (RECIST) or World Health Organization criteria (Postow et al., J. Clin. Oncol. 33 (2015), 1974-1982).

Case 4: Complete Clinical Remission of Stage IV Breast Cancer with Liver, Lung, Bone and Lymph Node Metastasis

The patient (56 y, female) initially presented with far advanced stage IV breast cancer pT3 pN2 M1 (bone, liver, lung) with Karnofsky index of 50% with serious neurological deficits from a large progressive skull metastasis which had started expanding and infiltrating the Dura Mater in spite of previous radiation.

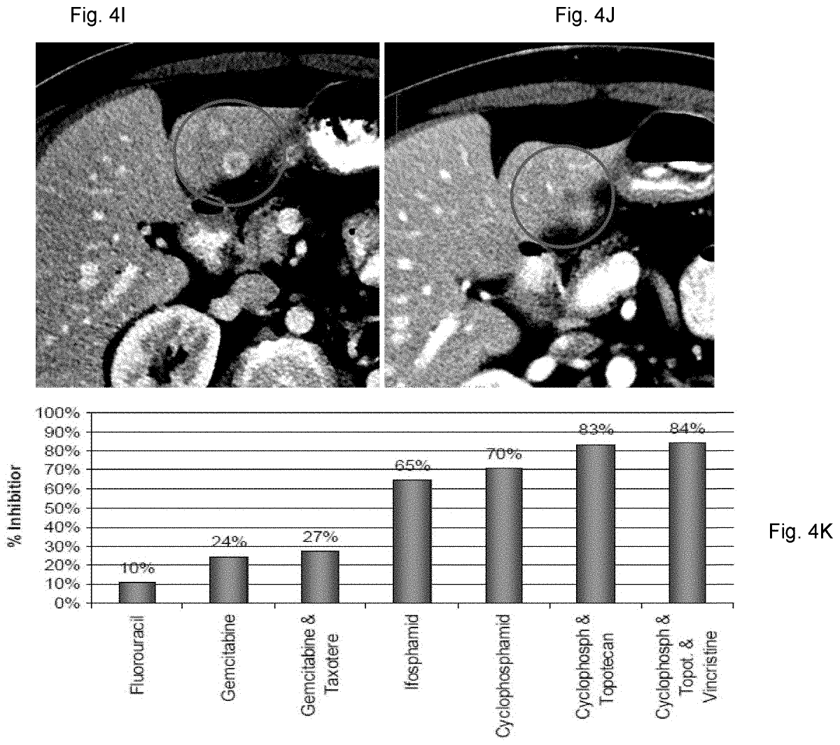

The patient underwent immunotherapy as described previously combining low-dose checkpoint inhibitor ipilimumab-nivolumab in combination with low dose interleukin (IL-2) treatment according to the present invention for four consecutive days parallel to local regional and whole-body hyperthermia. Additionally, low-dose metronomic chemotherapy was performed with Topotecan (0.5-1 mg/m²) and Capecitabine 1000 mg bid, 2w on/1w off following chemo sensitivity testing (FIG. 4K):

FIG. 4A-D shows the body core temperature of the cancer patient of case 4 at four exemplary consecutive days; FIGS. 4E-J show pictures (pre/after treatment) of skull (E, F), abdomen/pelvis (G, H) and chest (I, J) following therapy according to the present invention demonstrating complete remission; FIG. 4K shows inhibition of chemotherapeutic compounds for this patient.

1. A method for the induction of fever in the treatment of a cancer patient, said method comprising administering to the patient a combination of an (a) antibody that specifically binds to and inhibits Programmed Death-1 (PD-1) and/or an antibody that specifically binds to and inhibits Programmed Death-L1 (PDL1); (b) an antibody that specifically binds to and inhibits Cytotoxic T-Lymphocyte Antigen-4 (CTLA-4); and (c) Interleukin-2 (IL-2), wherein the body core temperature of said patient is kept at a temperature of 39.0° C. to 40.5° C. for at least 5 h per day for at least 4 consecutive days by a low dose (LD) IL-2 treatment, wherein the overall amount of IL-2 administered is in the range of 20 to 250 million units IL-2/week.

2. The method according to claim 1, wherein the body core temperature of said patient is kept at a temperature of 39.0° C. to 40.5° C. for at least 6 h per day for at least 4 consecutive days.

3. The method according to claim 1, wherein the body core temperature of said patient is kept at a temperature of 39.0° C. to 40.5° C. for at least 5 h per day for at least 6 consecutive days.

4. The method according to claim 1, wherein the body temperature of said patient is controlled by administration of IL-2.

5. The method according to claim 1, wherein the treatment does not include administration of an antipyretic.

6. The method according to claim 1, wherein IL-2 is administered by continuous administration of 100,000 to 1,000,000 units/kg body weight.

7. The method according to claim 1, wherein, independently of each other, each antibody is administered at a dosage ranging from 0.05 to 1 mg/kg body weight, at least once a week, for at least two weeks.

8. The method according to claim 1, wherein cyclophosphamide is administered additionally to said patient in an amount of 100 to 500 mg/m².

9. The method according to claim 1, wherein taurolidine is administered additionally to said patient.

10. The method according to claim 1, wherein the overall amount of IL-2 administered is in the range of 40 to 70 million units IL-2/week.

11. The method according to claim 1, wherein the overall amount of IL-2 administered is in the range of 40 to 200 million units IL-2/week.

12. The method according to claim 1, wherein IL-2 is administered to a patient in an amount sufficient to keep the body temperature of the patient at temperatures of of 39.5° C. to 40.5° C. for at least 5 h per day for at least 4 consecutive days.

13. The method according to claim 1, wherein IL-2 is administered to a patient in an amount sufficient to bring the body temperature of the patient at least once a day at a (maximum) temperature of between 39.5 to 41.0° C.

14. The method according to claim 1, wherein IL-2 is administered to a patient in an amount to keep the body temperature in the patient for at least 5 h for each of five consecutive days at a temperature above 39.5° C. by this low dose IL-2 treatment.

15. The method according to claim 1, wherein IL-2 is administered for one, two, three or four weeks.

16. The method according to claim 1, wherein IL-2 is administered in an amount of 5 to 50 million units as a daily dosage.

17. The method according to claim 1, wherein 40 to 100 million units of IL-2 are administered on the first day.

18. The method according to claim 1, wherein 40 to 100 million units IL-2 are administered on the first day, 20 to 50 million units IL-2 are administered on the second day, and 10 to 30 million units IL-2 are administered on the third, the fourth and the fifth days.

19. The method according to claim 1, where the antibody that binds to and inhibits PD-1 or PDL1 exhibits one or more of the following characteristics: (a) binds to human PD-1 with a KD of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo.

20. The method according to claim 1, where the antibody that binds to and inhibits CTLA-4 can bind to an epitope on human CTLA-4 so as to inhibit CTLA-4 from interacting with a human B7 counter receptor and binds to human CTLA-4 with a KD of 5×10⁻⁸ M or less.
